(19) United States
(12) Patent Application Publication
(10) Pub. No.: US 2010/0262257 A1
(43) Pub. Date: Oct. 14, 2010

(54) Joint Prosthesis for a Human Body and Associated Method

(76) Inventor: Patrick Cruchet, Miami Beach, FL (US)

Correspondence Address: ARENT FOX LLP, 1050 CONNECTICUT AVENUE, N.W., SUITE 400, WASHINGTON, DC, 20036, US

(21) Appl. No.: 12/420,167

(22) Filed: Apr. 8, 2009

(51) Int. Cl. A61F 2/32; A61F 2/36

(52) U.S. Cl. 623/22.29; 623/23.11

(57) ABSTRACT

The object of the present invention is a joint prosthesis for a human body including: a spherical head (1), an intermediate semi-spherical shell (2) in which the spherical head (1) is accommodated with a first clearance, a safety retaining ring (3), retaining the spherical head (1) in the intermediate shell (2), and an external shell (4) provided with a concave internal wall having the shape of a spherical cap, wherein the intermediate shell (2) is housed with a second clearance, with the concave internal wall having a local cavity (42) providing a storage for the lubricating liquid.

### TECHNICAL FIELD OF THE INVENTION

The invention relates to the field of implants or prostheses for the human body and more particularly a prosthesis for a joint such as the hip, or the shoulder, or a finger.

### STATE OF THE PRIOR ART

The prior art already knows above-knee and/or hip prostheses more particularly including implants. Such implants generally include a spindle for the impacted anchoring in an osseous zone of the thigh bone, as well as a head for the attachment to the hip.

The present invention more particularly concerns the head for the attachment to the hip or any other element of the same type.

A hip prosthesis including a semi-spherical head accommodated in a semi-spherical shell, which is itself accommodated in an external metallic element is known from patent U.S. Pat. No. 6,802,866. In addition, the metallic spindle connected to the spherical head includes a sleeve intended for limiting and damping the motion thereof with respect to the semi-spherical shell. Wear at the point of contact between the two elements is thus reduced.

In addition, the prior art includes the patent application US 2007/0038305 which discloses a prosthesis more particularly including a spherical head partially covered by an intermediate part made of plastic, which is itself accommodated in a metallic cup. Such joint significantly improves the torsional strength, although of reduced dimensions.

A prosthesis of the same type is also known from patent U.S. Pat. No. 7,393,362, although made of four parts; an additional part made of ceramic is provided, interposed between the spherical head and an intermediate part. Besides, a collar is provided for retaining the spherical head in the additional part. This concept enables a double mobility of the spindle anchored in the femur, with respect to the hip. The mobility of the prosthesis is thus significantly increased, while maintaining a good resistance.

This type of prostheses, however, demonstrated that it was liable to wear and age prematurely, in that the parts can deform, with the surfaces wearing in a non-uniform way. As a matter of fact, the weight, the efforts and the shocks which such a prosthesis is exposed to may be very severe, which shortens the service life thereof. Now, it is very important that the prostheses can be efficient for as long a time as possible, since the replacement thereof is of course not desirable, and not desired by the patient.

### EXPOSURE OF THE INVENTION

The invention aims at remedying the drawbacks of the state of the art, and more particularly at providing a prosthesis of the above-mentioned type, which has an improved lubrication between the composing elements thereof.

The aim is to extend the service life of prostheses, more particularly of so-called prostheses with double mobility.

For this purpose and according to a first aspect of the invention, a joint prosthesis for a human body is provided, including:
a spherical head,
an intermediate semi-spherical shell in which the spherical head is accommodated with a first clearance,
a safety retaining ring, retaining the spherical head in the intermediate shell,
an external shell provided with a concave internal wall having the shape of a spherical cap, wherein the intermediate shell is accommodated with a second clearance, with the concave internal wall having a local cavity providing a storage for a lubricating liquid.

The local cavity improves the lubrication between the intermediate shell and the external shell since, when the prosthesis is positioned, not only the cavity but the space between the two shells is filled with synovial liquid.

Preferably, the cavity is positioned in a distal zone with respect to the edges of the external shell. Such position makes it possible for the liquid in the cavity to be distributed uniformly, like a film, between the two shells.

In addition, it is advantageous for the internal wall of the external shell not to include sharp edges, more particularly on the perimeter of the local cavity, in order to avoid any wearing during the motions and to favour the circulation of the synovial liquid.

Thus, the local cavity has the shape of a spherical cap and at the junction thereof with the internal wall of the external shell, the angle (x between the tangent of said local cavity and the tangent of said internal wall, is between 0.5° and 45°. Wear between the two moving parts is reduced inside this range.

According to an advantageous characteristic, the cavity has a volume between 0.03 and 0.20% of the volume defined inside the surface of the external shell covering the intermediate shell. Such a volume provides a storage of synovial liquid for supplying the lubricating film between the two shells.

Besides, the clearance between the intermediate shell and the external shell is between about 0.10 and 0.25 mm. This dimension makes it possible to provide a stress-damping cushion between the two concerned elements. This contributes to the extension of the service life of the prosthesis.

In addition, the local cavity has the shape of a spherical cap and at the junction thereof with the internal wall of the external shell, the angle α between the tangent of said local cavity and the tangent of said internal wall, is between 0.5° and 45°. Wear between the two moving parts is reduced inside this range.

According to one embodiment of the invention, the edge of the external shell is extended by a cylindrical surface.

Advantageously, the retaining ring is made of a plastic material and the head is made of a ceramic material or a metal.

In addition, the intermediate shell may be made of a metallic, polyethylene or a ceramic material.

The external shell may be composed of the same materials.

The man skilled in the art will select the type of material to be used, as a function of economical, technical and/or other constraints.

According to another embodiment of the invention, the external shell may include several external fins positioned radially and in the vicinity of the circular edge thereof. This characteristic improves the anchoring of the external shell in the pelvis.

The present invention further relates to a method for replacing a part of a joint by a prosthesis, such as defined.

### BRIEF DESCRIPTION OF THE FIGURES

Other characteristics, details and advantages of the invention will appear upon reading the following description and referring to the appended figures, in which:

FIG. 1, is a cross-sectional view of a prosthesis according to a first embodiment of the invention;

FIG. 2, is a cross-sectional view of a prosthesis according to another embodiment of the invention;

FIG. 3, is a view, from inside, of the external shell; and

FIG. 4 is a cross-sectional diagram of the junction zone between the local cavity and the internal wall of the external shell.

For clarity, identical or similar elements are referenced with identical reference numbers on all the Figures.

### DETAILED DESCRIPTION OF ONE EMBODIMENT

FIG. 1 shows a longitudinal cross-section of a first embodiment of the invention. This Figure illustrates a spindle 0 which, in a known way, may be a femoral prosthesis, as a whole. The spindle 0 is crowned by a spherical, or semi-spherical head 1, or a head having a close shape. The head is generally made of a ceramic material, or of a metal or of a chromium-cobalt alloy.

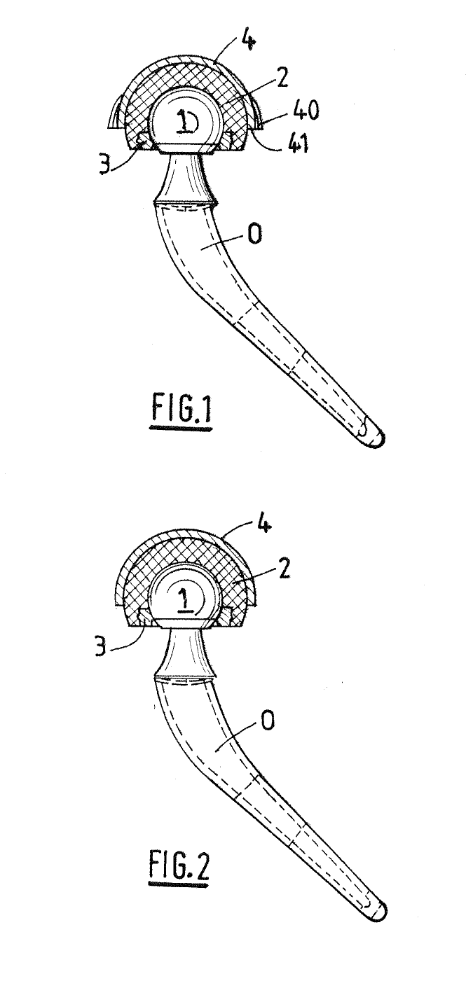

The head 1 is housed with a first clearance in a semi-spherical intermediate shell 2, wherein it is retained by a retaining ring 3 or an equivalent element. The intermediate shell 2 may be made of a metallic, polyethylene or a ceramic material. The head 1 is mounted with a first clearance in the shell 2. As an illustration, the first clearance is of the order of 0.15 micrometres to 0.40 micrometres. This clearance depends on the dimensions of the parts 1 and 2, on the nature thereof etc, and it will be selected by the man skilled in the art. The shell 2 covers most of the surface of the head 1 as illustrated in FIGS. 1 and 2.

In addition, the intermediate shell 2 is itself covered by an external shell 4 which it is accommodated in, while both elements rotate freely. This arrangement is known in itself, as a prosthesis with a double mobility. Additionally, in order to reinforce the connection of the external shell 4 to the patient's bone, fins 40 may be provided on the external wall of the external shell 4. The fins, for example, in the number of three, are angularly spaced by 120°, and positioned in the vicinity of the circular edge 41 of the shell 4.

The external shell 4 may be made of a ceramic material, or of a plastic material or it may even be metallic. Between the intermediate shell 2 and the external shell 4 is provided a clearance which can be between 0.10 and 0.30 mm.

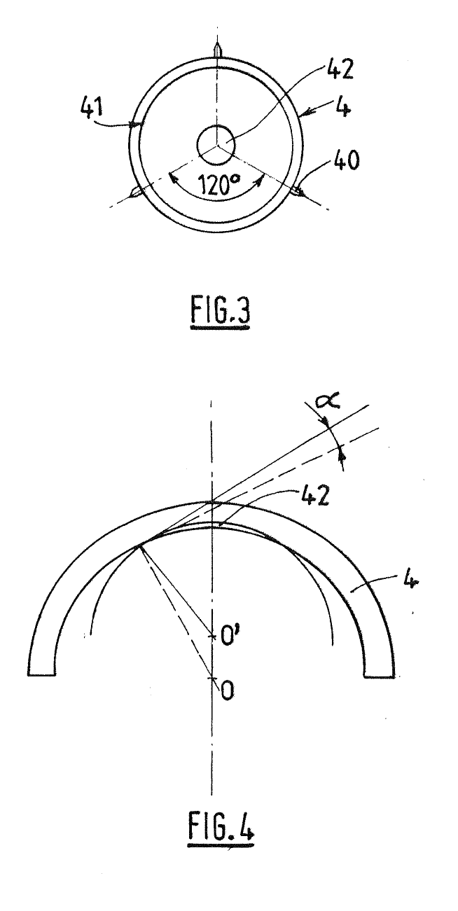

In addition, as can be seen more particularly in FIG. 3, a local cavity 42 is provided in the internal wall of the external shell 4; and this cavity is advantageously a storage for a lubricating liquid. More precisely, once the prosthesis is positioned in a bone of the human body, the local cavity 42 may be filled with synovial liquid, present and wetting the articular tissues. The local cavity 42 is preferably positioned as illustrated in FIG. 3, i.e. furthest from the circular edge 41 of the external shell 4, and centered with respect to said edge. Even though the element 4 does not have a totally and perfectly symmetrical shape, the local cavity 42 will be positioned as much as possible in a distal zone to the edge 41 thereof. Thus, in situ, the liquid present in the local cavity 42 can be easily and uniformly distributed in the space between the intermediate shell 2 and the external shell 4, thus actively and efficiently participating in the lubrication of the prosthesis. Such a lubrication not only facilitates the mobility of the parts, but it also reduces wear thereof.

The shape of the local cavity 42 may be semi-spherical, but it can also be a cap, with a maximum diameter between 15 and 25% of the external diameter of the shell 4, close to the edge 41 thereof. The local cavity 42 may have any other close shape, and the junction thereof with the internal wall of the external shell 4 advantageously has no sharp edge.

Anyway, the internal wall of the external shell 4 includes neither a sharp edge nor any protruding part; more particularly, the junction zone between the local cavity 42 and the remaining part of the internal wall of the external shell 4 includes no protruding part.

Preferably, a so-called incident angle α is defined between the tangent of said local cavity 42 and the tangent of said internal wall of the external shell at the junction of the two walls. FIG. 4 more particularly and more precisely illustrates such angle. More precisely, in the cross-section of FIG. 4, the internal wall of the external shell 4 is a circle, with a center 0 the radius of which is in dotted lines; in the same plane, the cavity 42 is limited by a circle with a center O′ the radius of which is in solid lines. At the junction thereof, the tangents of such two circles form an angle α defined between the dotted and solid lines. Such angle can advantageously be between 0.5° and 45°.

In addition, the volume defined by the local cavity 42 is very small with respect to the internal volume of the external shell 4. The ratio of the two volumes can be between 0.03 and 0.20%.

In addition, the surface condition of the local cavity 42 is preferably chosen to be of the order of 0.05 micrometres. It can be obtained through a specific mechanical polishing, for example followed by a lapping, followed by a polishing with a flannel cloth, if the part is made of metal. Great care is taken of this surface condition, as well as that of the internal wall of the semi-spherical element 4, so as to facilitate lubrication. The aim is improving the mobility of the external shell 4 with respect to the intermediate shell 2, and reducing wear of said parts.

Without leaving the scope of the invention, the external shell 4 may be extended by a cylindrical surface which extends from the equatorial plane thereof and which is then the edge 41. As a non limitative example, and considering such a shell 4 having an external diameter of about 50 mm, the height of the surface of the cylindrical end may be of the order of 1.5 mm.

A preferred application of the invention relates to hip prostheses, but the invention can also be used for replacing the joint in a shoulder or in a finger. Miniaturized prostheses cause no particular technical problems.

The invention aims at a method for replacing a part of a joint using a prosthesis such as defined here-above.

1. A joint prosthesis for a human body including:
a spherical head,
an intermediate semi-spherical shell in which the spherical head is accommodated with a first clearance,
a safety retaining ring, retaining the spherical head in the intermediate shell,
an external shell provided with a concave internal wall having the shape of a spherical cap, wherein the intermediate shell is housed with a second clearance, with the concave internal wall having a local cavity providing a storage for a lubricating liquid.

2. A prosthesis according to claim 1, wherein the external shell has an edge, with the local cavity being positioned in a distal zone with respect to the edge.

3. A prosthesis according to one of claims 1 or 2, wherein the internal wall of the external shell has no sharp edge.

4. A prosthesis according to one of claims 1 or 2, wherein the local cavity has a volume between 0.03 and 0.20% of the volume defined inside the surface of the external shell covering the intermediate shell.

5. A prosthesis according to one of claims l or 2, wherein the second clearance ranges between about 0.10 and 0.30 mm.

6. A prosthesis according to one of claims 1 or 2, wherein the local cavity has the shape of a spherical cap and in that, at the junction thereof with the internal wall of the external shell, the angle ox between the tangent of said local cavity and the tangent of said internal wall, ranges between 0.5° and 45°.

7. A prosthesis according to one of claims 1 or 2, wherein the edge of the external shell is extended by a cylindrical surface.

8. A prosthesis according to one of claims 1 or 2, wherein the retaining ring is made of a plastic material.

9. A prosthesis according to one of claims 1 or 2, wherein the head is made of a ceramic material.

10. A prosthesis according to one of claims 1 or 2, wherein the intermediate shell is made of a metallic or polyethylene (PET) material or of a ceramic material.

11. A prosthesis according to one of claims 1 or 2, wherein the external shell is made of a metallic or polyethylene (PET) material or of a ceramic material.

12. A prosthesis according to one of claims 1 or 2, wherein the external shell includes several fins on the outside, which radially extend in the vicinity of the circular edge thereof.

13. A method for replacing a part of a joint with a prosthesis according to any one of claims 1 to 3.
